(19) United States
(12) Patent Application Publication
(10) Pub. No.: US 2009/0069834 A1
(43) Pub. Date: Mar. 12, 2009

(54) AUXILIARY TOOL FOR FORMATION OF IMPLANT PRE-HOLE

(76) Inventor: Hiroshi Ohguchi, Gifu (JP)

Correspondence Address: SCHMEISER, OLSEN & WATTS, 22 CENTURY HILL DRIVE, SUITE 302, LATHAM, NY, 12110, US

(21) Appl. No.: 12/298,773

(22) PCT Filed: Apr. 5, 2007
(86) PCT No.: PCT/JP2007/057664

§ 371 Date: Oct. 28, 2008

(30) Foreign Application Priority Data
Apr. 28, 2006 (JP) 2006-125035
Sep. 13, 2006 (JP) 2006-247561

(51) Int. Cl. A61B 17/34

(52) U.S. Cl. 606/185

(57) ABSTRACT

Auxiliary tool provides an implant pre-hole smaller than the implant hole without requiring much experience or imposing much burden on patients during the implant pre-hole formation. In addition, implant pre-holes allow safe and easy formation of implant hole(s) at the next stage and further development of the implant technique. Consequently, persons with thinner bones who have given up implant treatment could have a chance to undergo implantation. The auxiliary tool 10 is to form implant pre-holes smaller than the diameter of implant holes for implant placement, which is comprised of implant pre-hole forming needles, 11a to 11e ranging from 0.3 to 1.4 mm in diameter.

### CROSS REFERENCE TO RELATED APPLICATION

The present invention claims priority under 35 U.S.C 371 to International Application No. PCT/JP2007/057664 filed Apr. 5, 2007, Japanese Application No.2006-125035 filed Apr. 28, 2006, and Japanese Application No.2006-247561 filed Sep. 13, 2006, which is incorporated herein by reference.

### FIELD OF THE INVENTION

This invention relates to, but not limited to, an auxiliary tool for formation of implant pre-hole which diameter is smaller than the diameter of an implant hole for implant treatment.

### DESCRIPTION OF THE RELATED ART

For persons who lose a tooth, there is an implant treatment which allows masticatory and aesthetic recovery through placing an artificial tooth root into the jawbone. The implant treatment is performed through placing a screw-type or cone-type titanium fixture into the jawbone. A hole to place this fixture is generally formed by drills or osteotomes as shown in FIG. 12.

Conventional techniques to form implant holes with drills or osteotomes are shown in the Japanese Patent Application No.2003-52721 (JP2003-52721A) and No.2006-61377 (JP2006-61377A).

### SUMMARY OF THE INVENTION

The JP2003-52721A suggests osteotomes for implant treatment in order to develop osteotomes for oral implant treatment safely usable also in the mandibular treatment, because conventional osteotomes for implant treatment have originally been developed for the maxillary implant surgery and involve a risk in the mandibular treatment. The osteotomes for oral implant treatment described in the JP2003-52721A have the bended working part below the rod-state working part of the osteotomes and a dimple for malleting on the bended back as shown in FIG. 13. As described in paragraph 0017 of the JP2003-52721A, the extremely-bended handle and working part allows operation without interference of the maxilla. In addition, due to the hard mandibular bone, it is desirable to sharpen the tip of osteotomes. Furthermore, in strong malleting, the dimple provided on the bended back allows safe operations without displacement during malleting.

Consequently, the osteotomes described in the JP2003-52721A are tools for strong malleting, so a dimple for malleting was provided on the bended back as shown in FIG. 13. Furthermore, according to the technique described in JP2003-52721A,
a) As an indication, the adaptable vertical bone height between the bone crest and the sinus floor must be 5 mm or more. The range of practicable sinus lift is to be within 4 mm.
b) The osteotome from 1.6 to 2.0 mm in diameter is initially used for tapping up to the depth from 1 to 2 mm above the sinus floor.
c) In increments, osteotomes with thicker diameter are used.
d) An appropriate amount of bone graft is inserted and is compressed by repeating slightly and malleting for sinus lift
e) The sinus floor is elevated by malleting up to the predetermined position (depth) while checking the depth scale.

When placing implants into the maxilla, the use of implant bodies thicker than 4 mm in diameter is desirable. When placing a 4 mm diameter implant, the smaller osteotome from 3.3 to 3.8 mm in diameter must be used to achieve sufficient primary stability and the 4 mm diameter implant body is placed by malleting.

The possible advantages of the treatment technique described in JP2003-52721A are as follows.
1) For the maxillary rough bone, the implant hole can be formed without bone drills.
2) Sinus lift can be achieved by limited osteotomes and instruments
3) Removal of bone is not required.
4) Compression (lateral pressure)/condensation, remodeling, and augmentation/ widening of the bone are possible.
5) Little damage to the bone.
6) Safe and easy operation.

However, the technique described in JP2003-52721A requires “malleting” as a condition, so it might place a tremendous burden on patients for the reasons described below. The diameter of the initially used osteotomes in the JP2003-52721A is 1.6 to 2.0 mm in diameter, which are based on the premise that the bone where implant will be placed should be soft and have a certain thickness. Consequently, it is considered that the implant placement into the harder or thinner bone is difficult. In addition, a harmful influence might be exerted on the jawbone, nerves or vessels during first boring.

The JP2006-61377A describes an implant hole forming jig which enables formation of the fixture hole with high-accuracy. The implant hole forming jig described in the JP2006-61377A comprises two or more osteotomes. As shown in FIG. 14, this jig allows for forming hole 12 to place Fixture 1 into Jawbone 13. This Fixture 1 comprises the cylindrical Smaller Diameter Part 2 and Larger Diameter Part 3 at its tip.

In the Implant Hole Forming Jig described in JP2006-61377A, the apical shape of the Fourth Osteotome 7 used finally has a shape continuing distally from the tip of the cylindrical First Shape Part 8 and Second Shape Part 9. This First Shape Part 8 is the same diameter or substantially the same as the above-mentioned Smaller Diameter Part 2. This Second Shape Part 9 is the same shape as the above-mentioned Larger Diameter Part 3. The apical shape of Third Osteotome 6 used in the previous step has a cylindrical shape of the same diameter or substantially the same as the above-mentioned Smaller Diameter Part 2 and is longer in axial length than the above-mentioned First Shape Part 8.

However, the technique shown in paragraph 0017 in JP2006-61377A, the smaller placement hole 12 is firstly formed by malleting First Osteotome 4 into Jawbone 13. Subsequently, the hole 12 is enlarged by malleting Second Osteotome 5 into Jawbone 13 and is formed into a cylindrical shape by malleting Third Osteotome 6 into Jawbone 13. Consequently, this technique might place a tremendous burden on patients due to malleting. Because the osteotome initially used in JP2006-61377A is about 2.1 mm in diameter, a harmful influence might be exerted on the jawbone, nerves or vessels during first boring. In other words, in both JP2003-52721A and JP2006-61377A, the basis of formation of implant holes is to use osteotomes with thicker diameter in increments. Consequently, since the operation is either malleting or boring, the shock might exert a significant effect on the patient's jawbone or body.

The smallest osteotomes in diameter to be initially used range from 1.6 mm (JP2003-52721A) to 2.1 mm (JP2006-61377A), which are based on the premise of placing implant into the softer bone and using drills. Consequently, it seems that implant placement into the harder or thinner bone is difficult. In addition, physical and psychological strain could impose a heavy burden on patients during the operation.

Although there are blood vessels as well as nerves in and surrounding the jawbone, the anatomic location of these blood vessels and nerves depends on the patient's constitution and the thickness of the jawbone varies among individuals. Implant is an excellent treatment maneuver for a person who lost his or her teeth. However, there are only a few dental surgeons who can perform implants; only approximately 3% dentists have focused on the implant technique. The main reason is its difficulties in operation.

What makes the implant maneuver difficult is the condition that blood vessels as well as nerves exist in and surrounding the jawbone, as described above. If the blood vessels or nerves are wounded, patients might not only feel considerable pain, but it might also be hard to heal the wound due to the occurrence of paralysis after surgery. The inventor considers that the major reason of these incidences is due to the diameter size of the osteotomes; even the smallest diameters of the osteotomes are from 1.6 mm (JP2003-52721A) to 2.1 mm (JP2006-61377A) used in the conventional technique. In other words, only a highly-skilled surgeon can use such thick “osteotomes” without widely hurting the nerves or blood vessels. Furthermore, if great forces such as “malleting” and “boring” were applied from the beginning, not only the wound of nerve or blood vessels, but also “fracture” or “transaction” could occur.

Furthermore, the above-described conventional technique such as implant holes are formed to place implants requires the following conditions: each thickness of the labial, palatal, and lingual cervical bones around the tooth root must be 1 mm or more, because the bone around the implant body having a certain level of thickness (1 mm or more) after implant placement allows for resisting attack by periodontopathic bacteria as well as bearing occlusal stress. Therefore, when a long time has elapsed since tooth loss, persons, people may not have enough bone to support the implant and also the implant hole due to the reduction in bone. Consequently, it is this fact that such persons cannot help giving up implant treatment.

Therefore, the inventor et al. have deliberated to develop an implant technique which not only requires less experience and imposes less burden on patients, but also confers a benefit on the patients who have given up implant treatment. As a result, we discovered that instead of direct formation of the implant hole, the formation of a pre-hole in advance might be the best way, and this was used in the current invention.

In accordance with at least one embodiment, there is provided an auxiliary tool that can form implant pre-holes smaller than implant holes for doctors without much experience and imposes fewer burdens on patients during pre-hole formation. In addition, the existence of implant pre-holes allows safe and easy formation of implant holes at the next stage and further development of the implant technique. Consequently, persons with the thinner bones who were previously ineligible for the implant treatment may have a chance to undergo the implant.

### Means for Solving the Problem

In accordance with at least one embodiment, there is provided the auxiliary tool comprising of; implant pre-hole forming needles between about 0.3 mm and about 1.4 mm in diameter for forming a implant pre-hole, these sizes are smaller than the implant hole. Thus, the invention described in claim 1 relates to Auxiliary Tool 10 not to directly form implant holes to place implants, but to form implant pre-holes smaller in diameter than them. This Auxiliary Tool 10 is comprised of multiple needles ranging between 0.3 mm and 1.4 mm in diameter at the Needle Part 13. These needles are used in order from 11a (The diameter of First implant pre- hole forming needle is smallest) to 11e (The diameter of Fifth implant pre-hole forming needle is largest) to form implant pre-holes for intended implant holes.

With Auxiliary Tool 10, the diameter of the Needle Part 13 must range from 0.3 mm to 1.4 mm because if the diameter of Needle Part 13 is smaller than the minimal diameter, 0.3 mm, it might not only make it difficult to form implant pre-holes, but could also increase the risk of the Needle Part 13 fracturing the inside of the bone. Conversely, if the diameter of Needle Part 13 is larger than the maximum diameter, 1.4 mm, operations on the thinner jawbone or harder bone become difficult, resulting in imposing a heavy physical and psychological strain on patients during the operation.

Because all implant pre-hole forming needles from first to fifth constituting the Auxiliary Tool 10 are extremely thin, these needles are basically used by squeezing them while rotating each body with the fingers. In other words, use of the Auxiliary Axis 17 shown in FIG. 1 or in the reamers, or the usage of physical forces (as described below), such as ultrasonic waves and laser beams is a possibility. However, these implant pre-hole forming needles from first to fifth are basically to be used by squeezing by hand. Consequently, implant pre-holes can be formed without the shock of the malleting or a boring type.

This Auxiliary Tool 10 is used as below: Firstly, as shown in FIG. 4, a small hole to insert different needles including First implant pre-hole forming needle 11a into the cortical bone located on the surface of the jawbone is drilled on the cortical bone over the jawbone or after the mucosal flap formation using a long-neck round bar. At this time, the Guide 20 shown in FIG. 10 may be used to determine the position of the hole to be formed

Next, as shown in FIG. 5, with a commonly used reamer, is drilling on the cancellous bone located medial to the mucosa to insert the tip of the First implant pre-hole forming needles 11a etc. So far, cracks or chips on the cancellous bone will not occur. In addition, since nerves and blood vessels exist in the back of the cancellous bone, these tissues will not be wounded.

Next, Auxiliary Tool 10 involved in the invention is used. First implant pre-hole forming needle 11a is inserted into the cancellous bone through the hole formed near the surface of the cortical and cancellous bones while squeezing in the needle. The First implant pre-hole forming needle 11a is 0.3 mm in diameter at the Needle Part 13. Therefore, a wound on the mucosa and jawbone is extremely minor and no pain is involved as well as no major bleeding. Consequently, compact bone can be formed through naturally pushing the cancellous bone around the First implant pre-hole forming needle 11a due to a diameter of 0.3 mm.

In particular, this First implant pre-hole forming needle 11a is 0.3 mm in diameter at its Needle Part 13 and is used while squeezing in. Therefore, if vessels and nerves are located from that point onward, this needle can avoid them and if the needle should hurt them, the wound is extremely minor. Hurt vessels and nerves heal early because a 0.3 mm diameter Needle Part 13 is extremely thin. In addition, compact bone can be formed through naturally pushing the cancellous bone around the First implant pre-hole forming needle 11a without harmful effects on the cancellous bone.

During operations with the First implant pre-hole forming needle 11a, the maxillary sinus is located from that point onward and even if the bone to be operated is low in height or thin in thickness, a implant pre-hole can be formed without harmful effects on the mucosa in the maxillary sinus because implant pre-hole formation with this First implant pre-hole forming needle 11a is performed by squeezing it in without the use of malleting or boring technique that gives a shock, only using an extremely thin needle. This is also the same as with implant pre-hole forming needle 11b etc. to be used in the next stage.

This method, in particular, is applicable to patients with thin or narrow bones who were previously ineligible for implant placement.

The use of this first implant pre-hole forming needle 11a allows for forming a hole that facilitates the use of the next second pilot docket forming needle 11b. For skilled dentists, the thickness of this hole can be easily known from sensing the invasion level of needle Part 13 with their fingertips and for unskilled dentists, from a scale graduated on needle Part 13.

Afterwards, the second implant pre-hole forming needle 11b is used with the first implant pre-hole forming needle 11a. This time, the second implant pre-hole forming needle 11b can relatively-smoothly penetrate as shown in FIG. 6 because the first implant pre-hole forming needle 11a “paves a way” as it is called. Of course, no adverse effects to vessels, nerves, and the cancellous bone are caused by this second implant pre-hole forming needle 11b since the first implant pre-hole forming needle 11a has already paved a “path.”

The implant pre-hole as shown on the left side in FIG. 7 is completed upon the performance of the above-described operation up to the final fifth implant pre-hole forming needle 11e. Auxiliary tool 10 with the best shape described below uses five step approaches with implant pre-hole forming needles from the first implant pre-hole forming needles 11a to the fifth implant pre-hole forming needle 11e. Of the 5 step approaches, some interim step approach(es) can be skipped. Dentists operating the surgery can freely decide which stage should be skipped, depending on the patient's age or jawbone condition.

As shown above, after the completion of the implant pre-hole, as shown in the right of the FIG. 7, with a commonly used osteotome described in JP2003-52721A and JP2006-61377A, an implant hole with inner diameter according to each implant placement becomes possible (see the right illustration of FIG. 7). Then, as shown in FIG. 8, the inside of the formed implant holes will be tailored to the configuration of the intended implant using a drill to place an implant body as shown in FIG. 9.

Furthermore, the currently devised auxiliary tool 10 can be applicable to patients with thin or narrow bones who could have not undergone the conventional implant holes forming technique due to the lack of bone thickness. The auxiliary tool 10 facilitates forming an implant pre-hole even for thin bones. Since in forming the implant pre-hole, the thinner bone can be expanded, utilizing the implant pre-hole, subsequent formation of the implant hole becomes possible. In other words, persons with the thinner bones who have given up implant treatment can benefit from the implant technique.

Like the first implant pre-hole forming needle 11a, extremely thin needles of Needle Part 13 should be disposable, because it is highly likely that the needle will be deformed by washing or under conditions of use.

Furthermore, all first to fifth implant pre-hole forming needles 11a to 11e constituting auxiliary tool 10 were basically used through squeezing them in while rotating each body part 14 by the fingers. In addition to this, after-mentioned physical forces, such as ultrasonic waves and laser beams can be utilized. The use of these physical forces allows for facilitating strength set or adjustment. Although “squeezing by hand” only depends on the operator's experience, the use of these physical forces allows for easily forming implant pre-holes with no shock, compared to the malleting or boring type.

From the results mentioned above, this auxiliary tool 10 involved in Claim 1 can form implant pre-holes smaller than implant holes without experience and imposes no burden on patients during implant pre-hole formation. Then, on the basis of these implant pre-holes, the implant holes can safely and easily be formed with osteotomes at the next stage.

Consequently, this auxiliary tool 10 involved in Claim 1 can form implant pre-holes smaller than implant holes without experience and imposes no burden on patients during implant pre-hole formation. In addition, the existence of implant pre-holes allows safe and easy formation of implant holes at the next stage and further development of the implant technique. Thus, persons with thinner bones who have given up implant treatment can benefit from the implant technique.

In accordance with at least one embodiment, there is the tips of implant pre-hole forming needles are formed into a conical shape, a bullet shape, a cusped shape, a cascade shape or a screw shape.

For auxiliary tool 10 involved in claim 2, as shown in the FIG. 2, the tips (tip 12) of the different implant pre-hole forming needles from 11a to 11e were sharpened into a conical, cannon, cusped, cascade or screw shape.

The example of tip l2 sharpened into a conical shape is shown in FIG. 2(1) and the example of tip 12 sharpened into a cannon shape in FIG. 2(2). And the example of tip 12 sharpened into a cusped shape in FIG. 2(3). The cusped shape means a head-sharpened form that is as if the flame of a burning candle was extended and the form was further sharpened compared to FIG. 2(1) and in FIG. 2(2). In addition, tip 12 having a dent on the edge is available.

Also, for implant pre-hole forming needles from 11a to 11e shown in FIG. 2(2), each tip may be cut into halves to form a flat surface, allowing the sharp side edge on the surface to grind the bone.

The example of tip l2 sharpened into a cascade shape is shown in FIG. 2(4). And the example of tip 12 sharpened into a cascade shape followed by rounding each cascade in FIG. 2(5). These tip shapes allow for making the cancellous bone more vertically and horizontally compact. The example of tip 12 sharpened into a screw shape is shown in FIG. 2(6). This tip shape would allow for further ensuring a squeezing operation.

As mentioned above, in either case, sharpening the tip 12 of different implant pre-hole forming needles constituting auxiliary tool 10 allows for ensuring invasion into the cancellous bone to form implant pre-holes by squeezing different implant pre-hole forming needles from first to fifth.

Consequently, this auxiliary tool 10 described in Claim 2 allows for providing the function similar to the one described in Claim 1 and ensuring a squeezing in operation.

In accordance with at least an embodiment, there is provided the body of the implant pre-hole forming needles having a portion selected from the group consisting of a anti-slip portion and a anti-rotation portion.

In addition, on the auxiliary tool 10 described in Claim 3, either anti-slips area 15 for the body 14 of each implant pre-hole forming needle (11a-11e) as shown in FIG. 2(1) or anti-rotation area 16 for the body 14 of the each implant pre-hole forming needle (11a-11e) as shown in FIG. 2(2) was formed.

The different implant pre-hole forming needles from 11a to 11e constituting auxiliary tool 10 must easily be able to be operated while rotating by fingers. Consequently, anti-slip area 15 shown in FIG. 2(1) allows for ensuring the squeezing operation. In addition, anti-rotation area 16 shown in FIG. 2(2) becomes effective while inserting each needle into auxiliary axis 17 to allow for ensuring a squeezing in operation during rotation of auxiliary axis 17.

As mentioned, this auxiliary tool 10 described in Claim 3 allows for ensuring a squeezing in operation as well as providing the function similar to one described in Claim 1 and 2.

As described above, in order to form holes in which implants will be placed, implant pre-holes smaller than the diameter of the implant holes will be formed using auxiliary tool 10 which is constitutionally characterized by comprising implant pre-hole forming needles, 11a to 11e ranging from 0.3 to 1.4 mm in diameter. With the auxiliary tool 10, implant pre-holes smaller than the implant holes can be formed without operator's much experience. The current invention can also provide the auxiliary tool 10 which further develops implant techniques with little burden on patients during implant pre-hole formation.

Also, the auxiliary tool 10 can easily use physical forces such as ultrasonic waves and laser beams and can be easily assembled onto a vibration generator 30 as shown in FIG. 11. In other words, by borrowing the physical forces or vibration from the vibration generator 30, the auxiliary tool 10 can easily form an implant pre-hole with less shock as compared with the malleting type or boring type.

From the results mentioned above, this auxiliary tool 10 involved in Claim 1 can form implant pre-holes smaller than implant holes without experience and imposes no burden on patients during implant pre-hole formation. Thus, on the basis of these implant pre-holes, the implant holes can safely and easily be formed with osteotomes at the next stage.

### BRIEF DESCRIPTION OF THE DRAWINGS

The embodiment will now be described, by way of example only, with reference to the accompanying drawings which are meant to be exemplary, not limiting, and wherein like elements are numbered alike in several Figures, in which:

FIG. 1 is a plan view of auxiliary tool 10 involved in the current invention.

FIG. 2 is a magnified figure of the chief parts of each first implant pre-hole forming needle constituting the same auxiliary tool. (1) the plan view of the needle having an anti-slip grip, (2) the plan view of the needle having an anti-rotation object (3)-(6) partially magnified plan view illustrating the shapes of the tips.

FIG. 3 shows the other auxiliary axis supporting the auxiliary tool, (1) front elevation view, (2) perspective view.

FIG. 4 is a fragmentary sectional view of the jawbone while drilling a hole on the cortical bone through the mucosa.

FIG. 5 is a fragmentary sectional view of jawbone during drilling a hole on the cortical bone.

FIG. 6 is a fragmentary sectional view of the jawbone during forming of an implant pre-hole on the cortical bone using the auxiliary tool involved in the current invention.

FIG. 7 is a fragmentary sectional view of the jawbone during making the implant pre-hole into an implant hole with the conventional technique.

FIG. 8 is a fragmentary sectional view of the jawbone while arranging the inside of the implant hole with the conventional technique.

FIG. 9 is a fragmentary sectional view of the jawbone during placement of the implant body in the implant hole using the conventional technique.

FIG. 10 is a top view illustrating guides, (1) a guide removed from the jawbone (mucosa surface), (2) the guide attached to the jawbone (mucosa surface)

FIG. 11 is a perspective view of the vibration generator providing vibration for the auxiliary tool involved in the current invention.

FIG. 12 is a plan view illustrating the conventional osteotome.

FIG. 13 is a perspective view of the osteotome described in the JP2003-52721A.

FIG. 14 is an enlarged plan view of an osteotome other than the conventional osteotome Description of the reference numerals

### DETAILED DESCRIPTION OF REFFERENCED EMBODIMENT

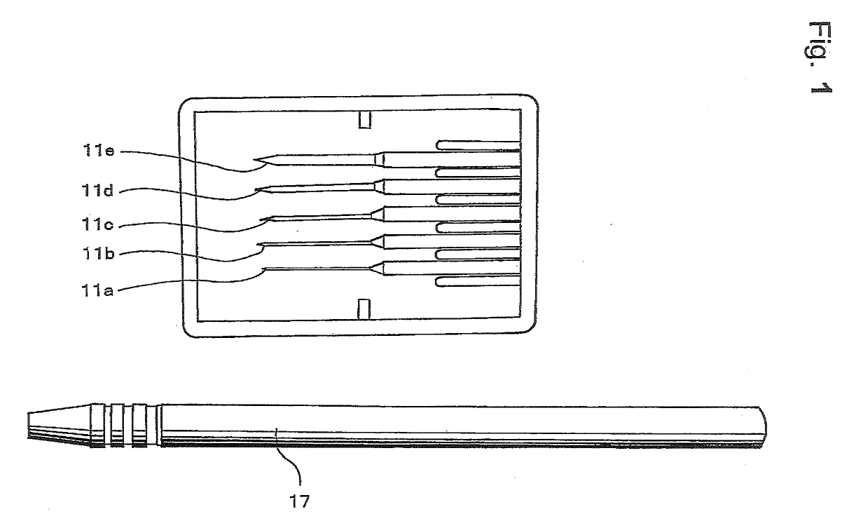

Next, for the invention involved in each claim organized as mentioned above, relates to the best shape of auxiliary tool 10 which will be explained below. The auxiliary tool 10 involved in the invention is shown in FIG. 1.

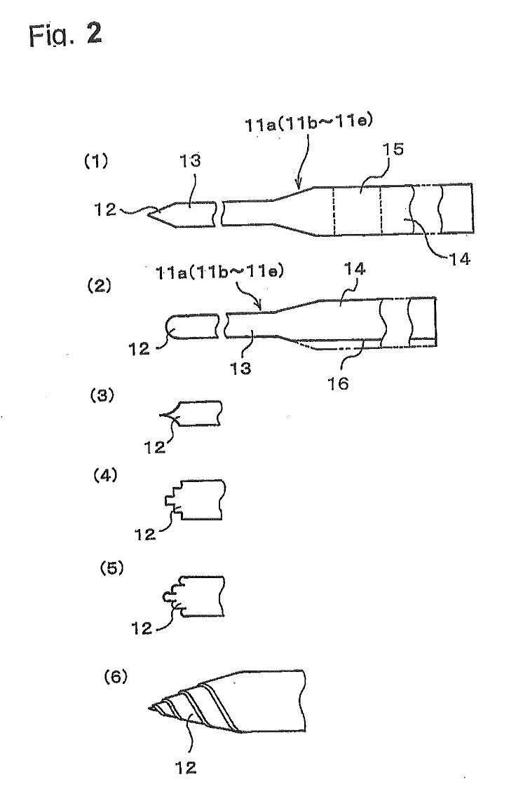

This auxiliary tool 10 comprises of five implant pre-hole forming needles form 11a to 11e (the best combination) and auxiliary axis 17 as shown in FIG. 2(1) and FIG. 2(2). The different implant pre-hole forming needles consist of tip 12, subsequent needle part 13, and body 14.

For the best shape, each needle part 13 of the first implant pre-hole forming needle 11a to the fifth implant pre-hole forming needle are defined as follows.
First implant pre-hole forming needle 11a; 0.5 mm in diameter
Second implant pre-hole forming needle 11b; 0.7 mm in diameter
Third implant pre-hole forming needle 11c; 0.9 mm in diameter
Fourth implant pre-hole forming needle 11d; 1.2 mm in diameter
Fifth implant pre-hole forming needle 11e; 1.4 mm in diameter
   The size of needle part 13 is ranges from 0.5 mm (smallest size) to 1.4 mm (largest size) in diameter and is of variable size. The number of needle parts is also decided freely within a range of the above mentioned sizes (from 0.5 mm to 1.4 mm). The smallest number of needles to be used will be two.

It is desirable to make these five implant pre-hole forming needles form 11a to 11e from metal. However, if tip 12 can be machined into various shapes as from (1) to (6) in FIG. 2 and rigidity that allows for squeezing tip 12 into the cancellous bone; can be ensured, resin materials may be used.

Since the different implant pre-hole forming needles from 11a to 11e are basically used while rotating each one with the fingers, Anti-slip area 15 must be provided on the surface of each Body 14 as shown in FIG. 2(1). This Anti-slip area 15 may be provided by forming many fine grooves such as serrations or attaching a rubber grip.

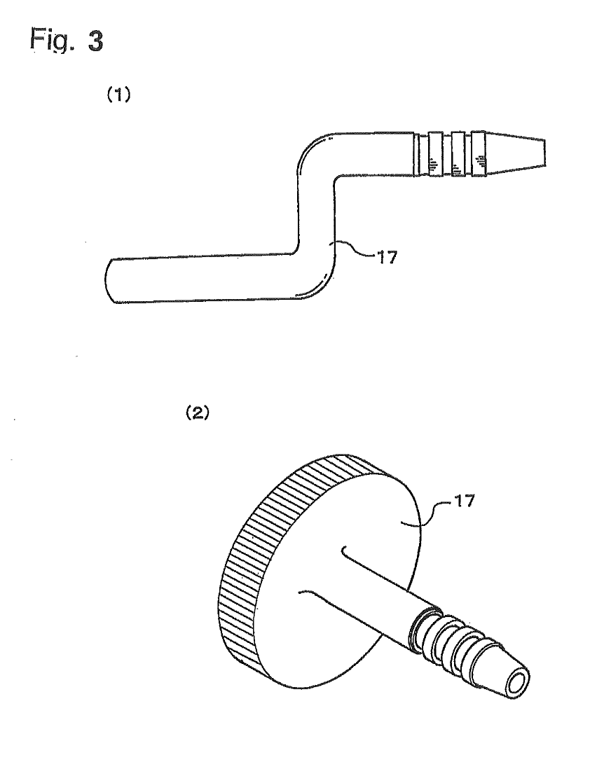

In addition, when these different implant pre-hole forming needles from 11a to 11e use Auxiliary Axis 17 shown in FIG. 1, in FIG. 3(1) or in FIG. 3(2), a semicircular hole on the inside of this Auxiliary Axis 17 can be formed. And then, Anti-rotation area 16 as shown in FIG. 2(2) is formed on each Body 14 to be inserted into this hole in order to fix Body 14 into Auxiliary Axis 17. This Auxiliary Axis 17 can be a linear shape as shown in FIG. 1, a crank shape as in FIG. 3(1) or a discoid shape as in FIG. 3(2), which will facilitate the formation of implant pre-holes for posterior teeth.

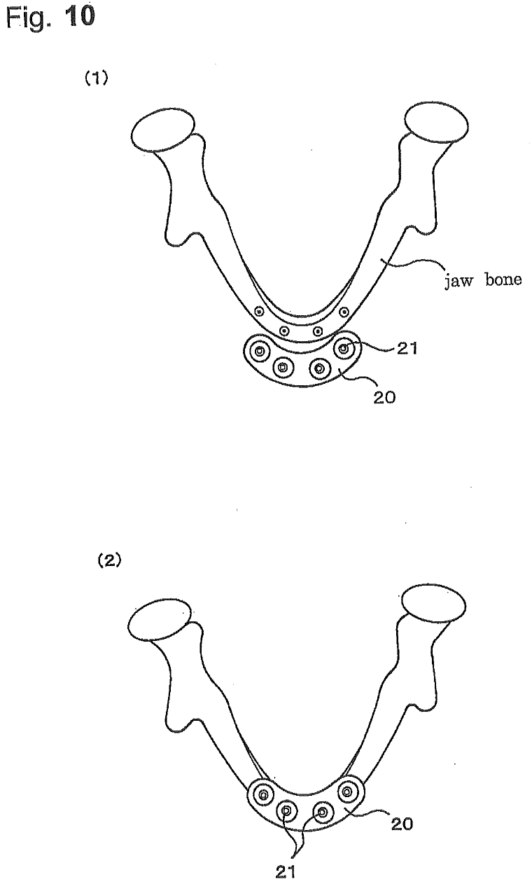

It is recommended to use Guide 20 as shown in FIG. 10 just before the use of this auxiliary tool 10. As shown FIG. 10(1) and (2), this guide 20 has guide holes 21; one more to help in determining the position to form an implant pre-hole into the jawbone and is preformed according to the patient's situation.

An example used auxiliary tool 10 as mentioned above-mentioned in a 34-year-old male patient. This patient had developed serious periodontal disease, so that implant placement into the left maxillary central tooth (hereinafter referred to as 1) was attempted for improving the oral hygiene environment and the occlusal relationship as well as providing stabilization of periodontal disease and tissue regeneration.

However, because this patient's tooth 1 had been lost for more than twenty years, alveolar bone resorption was significant. The bone thickness, 3 mm below from the alveolar crest, was 2.8 mm and 6 mm below from the alveolar crest, was 3.2 mm. Both of these were inadequate to place an implant. There is no doubt that application of the common technique shown in JP2003-52721A or JP2006-61377A would cause perforation.

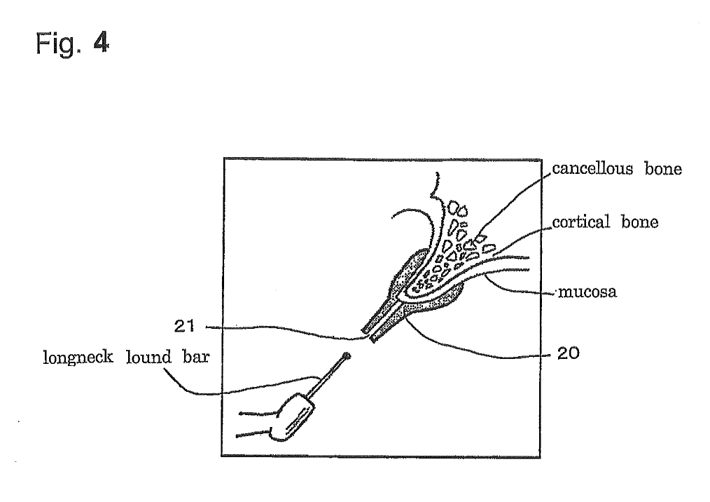

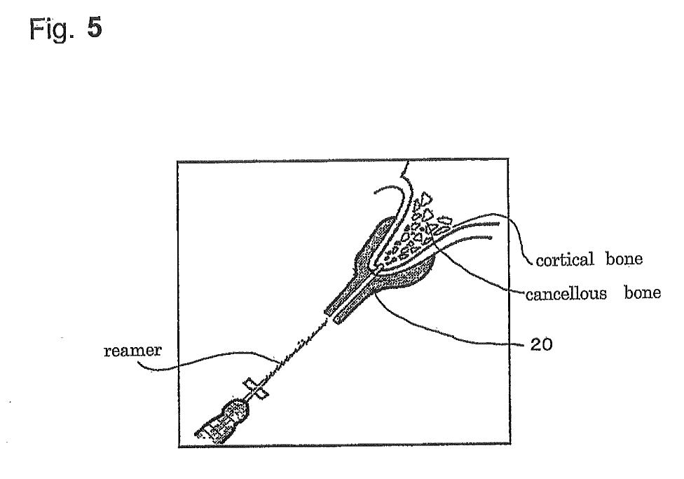

In order to attempt treatment with Auxiliary Tool 10 involved in the invention, the patient's mouth was cleaned and the formation of mucosal and periosteal flaps was minimized. Efforts were made to avoid invasion into them and infection during the operation. Auxiliary tool 10 was used only sensing with the fingers. As shown in FIG. 4, a minimum hole (0.5 mm diameter) was first drilled into the cortical bone at the bone crest with a round bar. And then, as shown in FIG. 5, an instrument was squeezed into the cancellous bone up to predetermined depth according to the specified implant length, 13 mm in this case, with a Zipperer reamer.

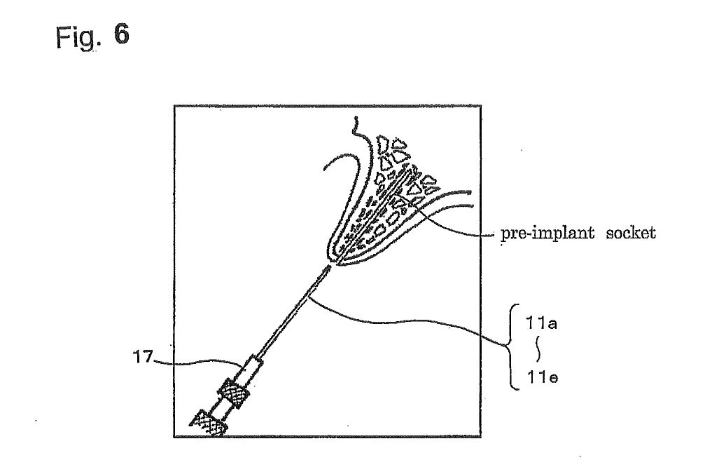

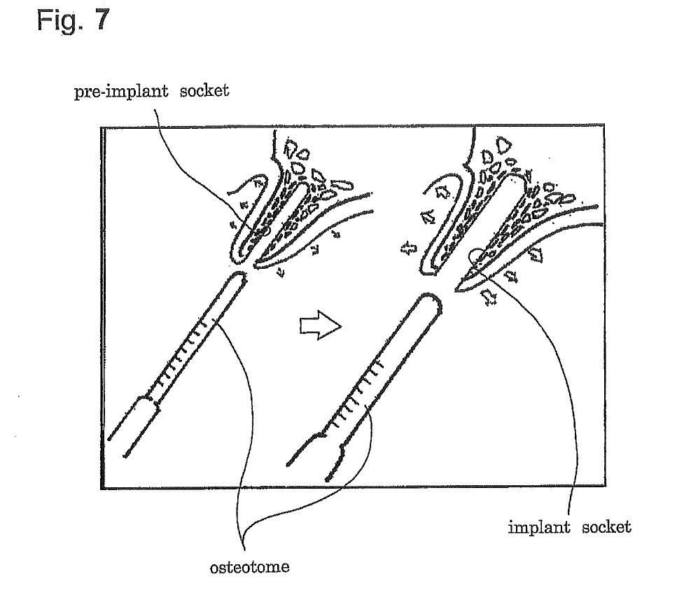

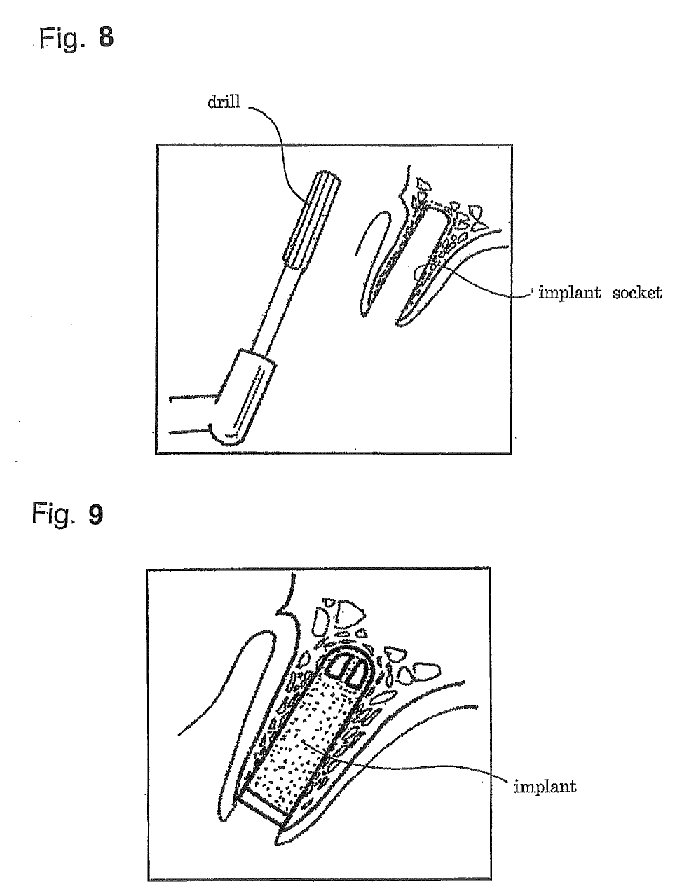

Then, as shown in FIG. 6, using Auxiliary Tool 10 in the order from 11a (First implant pre-hole forming needle smallest in diameter) to 11e (Fifth implant pre-hole forming needle largest in diameter), the implant pre-hole was expanded gradually. At the time, by pinching it with the fingers pulp of the other hand from the mucosal surface of the buccolingual side, the expansion of the body itself of the bone could be confirmed with the sensation of the fingers. Subsequently, as shown in FIG. 7, expansion of the hole could be expanded with a common osteotome, as shown in FIG. 9, and implant placement could be completed by adjusting it with a bone drill as shown in FIG. 8.

In this case, about 1 mm thick bone could be left on the buccal and palatal sides despite significant alveolar bone resorption and 2.8 mm thickness bone in the alveolar crest area. And then, the flap was replaced just by 2-place simple suturing.

Auxiliary tool 10 involved in the claimed invention is basically used through “squeezing by hand” as mentioned above. However, the addition of vibration generated from ultrasonic waves or compressed air or destructive power of laser beams to the forces of squeezing by hand can further improve operability.

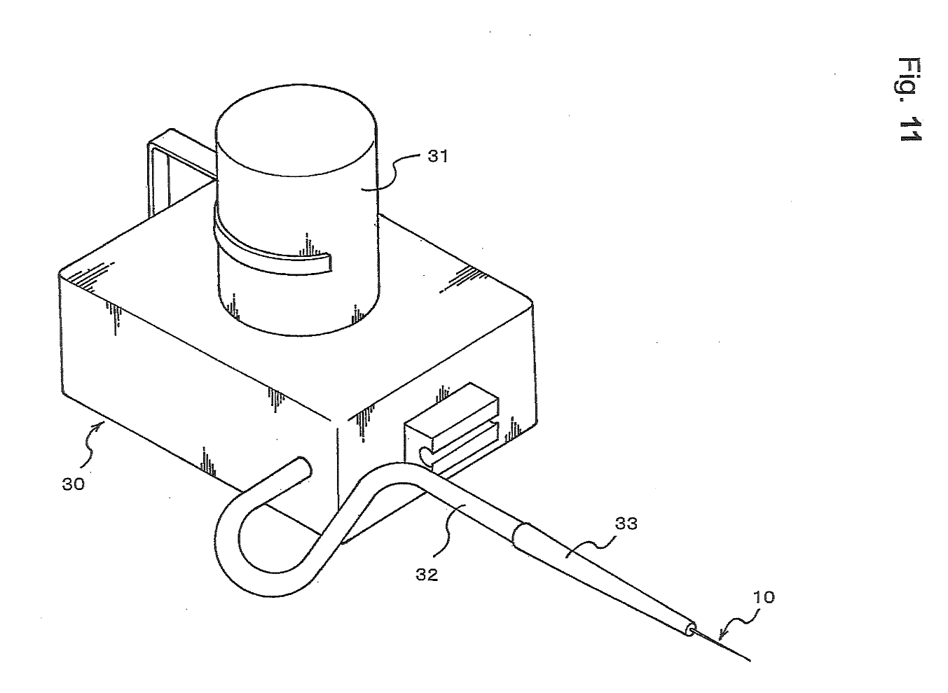

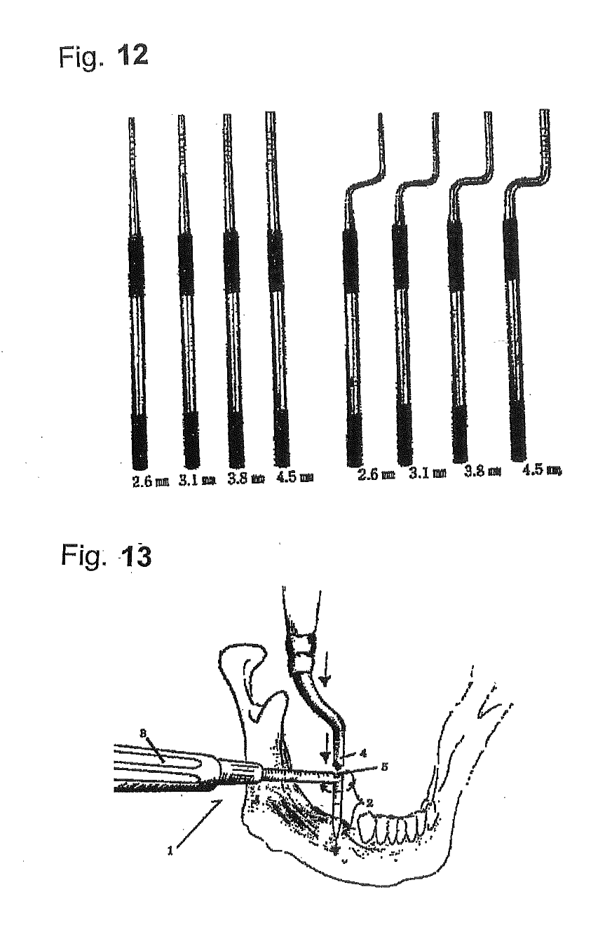

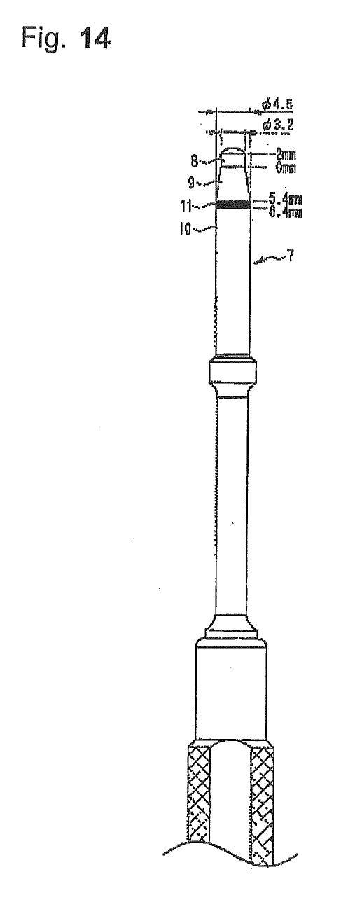

As shown in FIG. 11, the vibration generator 30 can be used with auxiliary tool 10 involved in the invention for ultrasonic vibration. This vibration generator 30 provides a function that produces electric vibration for generation of ultrasonic waves or compressed air. This electric signal or compressed air is supplied to hand-piece 33 through hose 32, by which ultrasonic or compressed-air vibration is generated. In other words, this vibration generator 30 has a function similar to ultrasonic scalers or air scalers used in dental treatment.

As shown in FIG. 11, it is recommended to attach water tank 31 to the vibration generator 30. Auxiliary tool 10 is a tool to go in the invention is a tool for the mouth to work while cleaning it. Therefore, water or physiological saline is stored in this water tank 31, from which a drop or mist of water or physiological saline is supplied into the tip of auxiliary tool 10 through the above-mentioned hose 32. Also, this vibration generator 30 using ultrasonic waves builds an ultrasonic-generating resonant rod in its hand-piece 33.

The way to attach auxiliary tool 10 to hand-piece 33 of vibration generator 30 is adjusting the above-mentioned body part 14 or anti-rotation 16 of the proposed auxiliary tool 10 to the suitable shape of the attachment of hand-piece 33. Preparation of adapter connecting hand-piece 33 of vibration generator 30 to auxiliary tool 10 is also effective so that auxiliary tool 10 can be attached to and removed from any shapes of vibration generator 30.

A vibration generator 30 which can generate not vibrations but a laser beam through the tip of auxiliary tool 10, can be applied. In that case, a hole through which the laser beam runs will be needed inside of auxiliary tool 10. However, with the recent technique of an auxiliary tool, with a diameter of 0.5 mm or more becomes available.

An Auxiliary tool 10 configured as mentioned above will be helpful for implant placement to fix artificial teeth in those who have lost teeth. In addition it is considered that auxiliary tool 10 may substantially contribute to dental practices.

Although implanting is a very beneficial technique, only 3% of dentists are currently utilizing the technique in earnest, according to statistics. The main reason is as mentioned in the section of “conventional technique” that the conventional technique requires a lot of experience and has caused various malpractices.

However, the implant technique utilizing the current invention does not require much experience, and rarely causes harm to the blood vessels, nerves, or the cancellous bone. Therefore, this implant placement technique can be widely used, even in persons with a thinner bone who have given up hope of implant treatment. The technique is also expected to substantially contribute to dental practices.

While the description above refers to particular embodiment of the present invention, it will be understood that many modifications may be made departing from the spirit thereof.

The accompanying claims are intended to cover such modifications as would fall within the true scope and spirit of the present invention.

The presently described embodiments are therefore to be considered in all respects as illustrative and restrictive, the scope of the invention being indicated by the appended claims, rather than the foregoing description, and all changes which come within the meaning and range of equivalency of the claims are therefore intended to be embraced to be embraced therein.

1. An auxiliary tool comprising;
pre-implant hole forming needles are between about 0.3 mm and about 1.4 mm in diameter for forming a pre-implant hole which size is smaller than the implant hole.

2. The auxiliary tool according to claim 1,
wherein the tips of pre-implant socket forming needles are formed a conical shape, a bullet shape, a cusped shape, a cascade shape or a screw shape.

3. The auxiliary tool according to claim 1,
wherein the body of the pre-implant socket forming needles having a portion selected from the group consisting of a anti-slip portion and a anti-rotation portion.

4. The auxiliary tool according to claim 2,
wherein the body of the implant pre-hole forming needles having a portion selected from the group consisting of a anti-slip portion and a anti-rotation portion.
